(19) United States
(12) Patent Application Publication
(10) Pub. No.: US 2011/0082221 A1
(43) Pub. Date: Apr. 7, 2011

(54) IN SITU GELLING SYSTEMS AS SUSTAINED DELIVERY FOR FRONT OF EYE

(76) Inventors: Claire Haug, Saint-Louis (FR); Stephane Jonat, Blotzheim (FR)

(21) Appl. No.: 12/996,436

(22) PCT Filed: Jun. 11, 2009
(86) PCT No.: PCT/EP2009/057250

§ 371 Date: Dec. 6, 2010

(30) Foreign Application Priority Data
Jun. 12, 2008 (EP) 08158152.2

(51) Int. Cl. A61K 47/36; A61P 27/02

(52) U.S. Cl. 514/779

(57) ABSTRACT

An ophthalmic formulation particularly well suited for use as a delivery vehicle in the sustained delivery of ophthalmic active agents to the eye. The formulations comprise an alginate, wherein the guluronic acid content is in a range of about 35 percent to about 45 percent, and an excipient, preferably gellan gum or scleroglucan, with an active agent dissolved or suspended therein that together provide an in situ-gelling system for use in the eye.

### FIELD OF THE INVENTION

This invention relates to extended release formulations for use in the delivery of an active agent to the eye.

### BACKGROUND

Upon instillation of an ophthalmic solution to the eye, most of the instilled fluid is lost due to drainage of excess fluid, and dilution and elimination of the solution by tears. Increasing ocular bioavailability has been the driving force in developing new approaches for ocular delivery of ophthalmic formulations. These approaches include the use of ointments, implants, and gels. Each of these approaches, however, has drawbacks. For instance, application of eye ointments may blur vision. Solid implants are often uncomfortable, and as a result patient compliance is low. Gels cause stickiness of the eyelid, and as a result are not widely accepted.

Recent efforts have begun utilizing phase-transition systems to deliver ophthalmic formulations. Such systems may be applied to the eye in a liquid, form and only once in the cul de sac of the eye they shift to a gel phase. Several such phase-transfer systems have been reported, with the change in phase being dependent on various factors, such as temperature, pH, or anion concentration. Polysaccharides are well suited for such systems because of their ability to retain water, while swelling to form a hydrogel. Alginate is a polysaccharide with fast gelling properties; forming gels strong enough to be suitable for many industrial and medical applications.

Use of alginates as in situ forming gels for ophthalmic drug delivery is known and described in U.S. Pat. No. 5,776,445. Alginate is a block-copolymer containing two kinds of homopolymeric blocks, of β-D mannuronic acid (M-M) blocks and α-L-guluronic acid (G-G) blocks together with blocks with alternating sequence (M-G). The affinity of alginates for divalent metal ions increases with increasing content of L-guluronic acid residues in the alginates. The gelling properties and resulting matrix are a result of the high guluronic acid content; formulations require a guluronic acid content of at least 50%.

Specific problems are present in the above, and other alginate in situ gelling systems. Although alginate is well suited for ophthalmic use, in that it is biocompatible and biodegradable, it does not allow for prolonged release of ophthalmic therapeutic agents on the front of the eye. Ocular bioavailability, therefore, is not improved by current alginate in situ-gelling systems.

Moreover, modified alginate phase-transition systems, such as gellan gum formulations as those described in Balasubramaniam et al., Drug Deliv, 10:185-191 (2003) show a maximum duration of drug release at eight hours. Thus, a patient is still required to apply a liquid form of the alginate solution to the cul de sac of the eye by eyedropper in conjunction with the gellan gum formulations, depending on the therapeutic agent involved, which is inconvenient for patients, thus leading to poor patient compliance.

### SUMMARY

Described herein is an alginate in situ-gelling vehicle and system that provides sustained delivery of an active agent—such as a drug or other therapeutic agent—to ocular tissue over an extended period of time, of up to 24 hours or more.

In one embodiment, alginate is combined with an excipient to form an in situ-gelling system. In another embodiment, alginate is present with a guluronic acid content in a range of about 35% to 45%. In one embodiment an excipient is scleroglucan or gellan gum. In yet another embodiment, alginate in combination with an excipient is incorporated together with a pharmaceutically active agent in order to form an in situ-gelling vehicle that provides sustained, extended release of the active agent. Ideal formulations of the in situ-gelling vehicle permit minimal instillation of the gel system once a day or once a week, depending on the active agent used.

In one embodiment, it has been discovered that the addition of gellan gum to an alginate containing about 35% to 45% guluronic acid provides an in situ-forming gel leading to a prolonged release of an active agent from the gelling vehicle in vitro and in vivo. It has also been discovered that the addition of scleroglucan to alginate containing about 35% to 45% guluronic acid provides an in situ-forming gel that improves release of drugs compared to conventional alginate in vitro and in vivo. An active agent can be either dissolved (hydrophilic) or suspended (hydrophobic), thereby becoming “entrapped” or incorporated in a gellan gum/alginate in situ-gelling vehicle or a scleroglucan/alginate in situ-gelling vehicle for sustained drug delivery.

In another embodiment, the in situ-gelling vehicle provides a drug delivery system for a wide range of treatment and is especially beneficial for delivering active agents over an extended period of time for a local treatment at the front of the eye, for example, dry eye, inflammatory reactions, microbial infections, as well as to facilitate the delivery of pharmaceutically active agents to the eye for the treatment of eye diseases, such as glaucoma.

In one embodiment, in situ-gelling vehicles as disclosed herein, are easily applied in the cul de sac of the eye, comparable to an eye drop. Formulations are viscous liquids prior to instillation in the cul de sac and undergo a phase transition from liquid to gel upon contact with anionic lachrymal fluid. Upon gellation, the in situ-gelling vehicle maintains integrity without dissolving or eroding for a prolonged period of time to facilitate sustained release of active agent to the eye surface and/or ocular tissue, depending on the absorptive properties of the agent.

The foregoing summary provides an exemplary overview of some aspects of the invention. It is not intended to be extensive, or absolutely require any key/critical elements of the invention.

### BRIEF DESCRIPTION OF THE DRAWINGS

The foregoing summary, as well as the following detailed description is explained with reference to the accompanying drawings. It should be understood, however, that the invention is not limited to the precise arrangements and instrumentalities shown.

FIG. 1 shows the extended release profile of 0.5% fluorescein ISGV formulations of one embodiment of the invention in vitro.

FIG. 2 shows the extended release profile of 0.5% fluorescein ISGV formulations of one embodiment of the invention in vivo using a rabbit model.

FIG. 3 shows the extended release profile of 3% ASM 981 ISGV formulations of another embodiment of the invention in vitro.

### DETAILED DESCRIPTION

The formulations described herein are for ocular use and comprise a sodium alginate with a guluronic acid content of about 35% to 45% as a gelling polymer in combination with an excipient, preferably a scleroglucan or a gellan gum, and a therapeutically active agent incorporated (dissolved or suspended) therein.

In one embodiment, in situ-gelling vehicles of alginate in combination with excipient are particularly useful for delivery of active agents over a prolonged period of time to the front of the eye or ocular tissue of a patient in need thereof, in a sustained, controlled manner for up to, and beyond, a 24 hour period.

In another embodiment, in situ gelling vehicles in an ophthalmic formulation comprising a sodium alginate, wherein the guluronic acid content is in a range of 35 percent to 45 percent, and an excipient in a gelling combination provide a method for treating disorders of the eye.

### DEFINITIONS

In describing and claiming the in situ-gelling system and method, the following terminology will be used in accordance with the definitions set forth below.

As used herein, “formulation” and “composition” may be used interchangeably and refer to a combination of two or more elements, or substances. In some embodiments a composition may include an active agent, an additional excipient, or a carrier to enhance delivery or gel formation.

“Active agent” as used herein refers to any therapeutically beneficial compound or pharmaceutical agent capable of being incorporated (dissolved or suspended, depending on solubility of the active agent) in the in situ-gelling vehicles. Suitable active gents include but are not limited to: glaucoma agents; anti-bacterial agents; anti-infective agents; hydrophobic and hydrophilic agents, and other active agents that may become apparent to one skilled in the art upon the benefit of this disclosure.

As used herein, “effective amount,” refers to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating a condition for which the active agent is known to be effective. The determination of an effective amount is well within the ordinary skill in the art of pharmaceutical sciences and medicine.

As used herein, “carrier” or “inert carrier” refers to a substance with which a drug may be combined to achieve a specific dosage formulation for delivery to a subject. In some embodiments, the carriers used may or may not enhance drug delivery. As a general principle, carriers must not react with the drug in a manner which substantially degrades or otherwise adversely affects the drug, except that carriers may react with a drug to prevent it from exerting a therapeutic effect until the drug is released from the carrier. Further, the carrier, or at least a portion thereof must be suitable for administration into a subject along with the drug. Additionally, the carrier may be used to increase the solubility of the drug, and thus act as a solubilizer.

As used herein, “eye”, “front of eye” and “ocular” refers to the peripheral visual organ of a subject.

As used herein, “subject” and “patient” are used interchangeably and refer to a mammal that may benefit from the administration of a composition or method as recited herein. Most often, the patient will be a human but can be of other animals such as dogs, cats, and horses.

As used herein, “administration,” and “administering” refer to the manner in which an active agent, or composition containing such, is presented to a subject.

As used herein, “about 35% to 45%” refers to the approximate percentage of guluronic acid compared to mannuronic acid of sodium alginate. An alginate comprised of about 35% guluronic acid, for example, by definition will also be comprised of about 65% mannuronic acid. It will be understood to one skilled in the art that “about” 35% refers to an amount sufficiently close to the amount, but may be, due to imprecision in methods of measurements, slightly less or more than the amount.

Reference herein to “one embodiment”, “an embodiment”, or similar formulations herein, means that a particular feature, structure, operation, or characteristic described in connection with the embodiment, is included in at least one embodiment of the invention. Thus, the appearances of such phrases or formulations herein are not necessarily all referring to the same embodiment.

Furthermore, various particular features, structures, operations, or characteristics may be combined in any suitable manner in one or more embodiments.

### In Situ-Gelling System

The optimized in situ-gelling system formulations described herein result in a drug delivery mechanism well suited for prolonged release of up to 24 hours of a therapeutically active agent further comprising a hydrophilic or hydrophobic drug substance, both in vitro and in vivo, when compared to classical alginate in situ gelling systems.

“In situ-gelling vehicles” (ISGVs) or “in situ-gelling system” (ISGS) as used herein refer to formulations comprised of the in situ-gel-forming polymer sodium alginate, with a guluronic acid content of about 35% to 45%, in combination with an excipient, ideally with a bioadhesive compound, such as scleroglucan, or another in situ gelling polymer, such as gellan gum, and a therapeutically active agent dissolved or suspended therein, suitable for use in sustained delivery of the therapeutically active agent to the front of the eye or ocular tissue of a patient in need thereof.

ISGV formulations are biocompatible. Thus, because they can interact with animal tissue without deleterious immunological effects, any active agent or molecule deliverable to a patient could be incorporated into a biocompatible ISGV for delivery to the front of the eye.

Formulations of one embodiment of the invention include an alginate together with an excipient, wherein the guluronic acid content of the alginate is in the range of about 35 percent to 45 percent, when compared to the percentage of mannuronic acid of the alginate. Thus, ideal formulations have a higher percentage of mannuronic acid to guluronic acid, unlike classical alginate systems previously shown for ocular use that require a minimal guluronic content of 50%. It has been discovered that higher mannuronic acid content substantially affects gelling properties and scaffolding of the in situ-gelling system, and helps provide the extended release profiles seen in the accompanying figures.

Suitable alginates include sodium alginate, such as available from FMC biopolymer (Philadelphia, Pa., USA). Concentration of alginate in suitable formulations ranges from 0.5 percent to 3 percent by weight. As appreciated by those skilled in the art, however, other suitable alginates may be used, and from different manufactures.

Suitable excipients include bioadhesive compounds, such as scleroglucan, or gelling polymers, such as gellan gum, which in combination with alginate provide sustained, extended delivery of active agent when compared to in situ-gelling systems comprised of alginate alone.

Suitable gellan gum excipients include Gelrite® (such as available from Sigma, St. Louis, Mo., USA), but other suitable gellan gum can be used in the ISGVs. In one embodiment, formulations comprise gellan gum in a concentration range of 0.015 percent to 0.06 percent, ideally 0.03 percent.

Suitable scleroglucan excipients include tinocare GL® 1% (such as available from Ciba Specialty Chemicals Corp., Tarrytown, N.Y., USA). In one embodiment, formulations comprise scleroglucan in a concentration range of 0.25 percent to 0.5 percent, ideally 0.25 percent.

In one embodiment, in situ-gelling vehicles are formulated with adequate viscosity for application in the cul de sac (conjuctival sac) of the eye. Formulations are viscous, i.e. “non-gelled”, prior to contact with lachrymal fluid of the eye. Gelling occurs rapidly, within minutes of applications and the gelled formulation provides a stable, safe, delivery mechanism for active agent to the eye over a time period of hours or days.

The ISGVs formulations are useful for drug delivery for a wide range of treatments and beneficial for delivery of active agents for local treatment at the front of the eye, for example, dry eye, inflammatory reactions, microbial infections, as well as to support the delivery of therapeutics to the eye in the treatment of glaucoma.

In one embodiment, ISGVs provide a method for treating a disorder of the eye, such as glaucoma, in a patient in need. In one embodiment, ISGVs are administered in a liquid ophthalmic formulation comprising an alginate, wherein the alginate has a guluronic acid content of about 35 percent to about 45 percent and an excipient, together with a therapeutically effective amount of an active agent for treating glaucoma. In another embodiment, instillation in the cul de sac of the eye initiates gelling of the ophthalmic formulation, resulting in controlled release of the active agent from the gelled formulation to ocular tissue of the eye.

One skilled in the art will recognize after having the benefit of this disclosure, that a wide variety of other suitable active agents may be formulated in the manner disclosed herein. Thus, the suggested agents discussed above are representative only.

In addition to their usefulness in delivering therapeutic agents to the eye, in another embodiment, ISGVs are also useful without a therapeutic active agent for preventing dryness of the eye. Dryness of the eye is often associated with or triggered by aging, environmental irritants, clinical conditions, such as malnutrition or clinical treatments, such as dryness of the eye seen with cancer patients undergoing chemotherapy.

In yet another embodiment, the formulations may further comprise other ophthalmically acceptable agents including buffers, preservatives, tonicity agents, and disinfecting agents.

Reference is now made to the following examples. These examples are provided for the purpose of illustration only and should in no way be construed as being limited to these examples but rather should be construed to encompass any and all variations, which become evident as a result of the teaching provided herein.

### EXAMPLE 1

The following procedure was utilized to test the effectiveness of the formulations of one embodiment for delivery of a hydrophilic active agent in vitro.

Optimized formulations included test formulations of ISGVs comprising alginate (1.5%)/gellan gum (0.03%) with 0.5% fluorescein (as the active agent), and comprising alginate(1.5%)/scleroglucan(0.25%) with 0.5% fluorescein (as active agent), which were prepared in accordance with the protocol at Example 3.

ISGVs containing 0.5% fluorescein were incubated in aqueous release medium and underwent periodic analysis for dissolution by RP HPLC with ultra-violet detection at 225 nm. Release medium was comprised of simulated tear fluid containing glucose, NaHCO₃, adenosine and glutathione. Samples of 100 μl from reference solution and test solution were aliquoted with a Rainin pipette and tested by HPLC. Equipment included an Agilent 1100 Series apparatus with a Nulceosil 100-5 C18 column 250 mm in length, with an internal diameter of 4.6 mm and a flow rate of 1.0 ml/min through the column. HPLC vials were Infochroma with inserts and the dissolution vessel was a 30 ml glass bottle with screw cap.

A system suitability test was performed prior to dissolution. Each batch contained between 3 and 6 samples. Samples were shaken at 60 rpm in circular motion with about 1 cm radius in a test medium of simulated tear fluid. Incubation involved filling the dissolution vessel with 30 ml test medium and tempering to 37° C.

The fluorescein reference was weighed to about 180 mg accurately to 0.1 mg with an analytical balance in a weighing pan and then transferred in the dissolution vessel. The alginate containing sample was solidified within the weighing pan upon contact with the test medium. The vessel was closed and placed on a lab shaker in an oven. Samples of 100 μl test medium were then transferred into HPLC vials and tested.

Chromatograms of a reference solution were compared to test solution and peak areas were calculated to provide a release profile. Evaluation to determine the peak area of fluorescein in the chromatograms of test solution and reference solution was performed as follows:
Calculation M=fluorescein released in mg/100 mg ISGS

$$M = \frac{{PA}_{T} \times {m}_{R} \times {C}_{R} \times {V}_{T} \times {V}_{D}}{{PA}_{R} \times {m}_{T} \times {V}_{RS} \times {V}_{R}}$$

Where
PA_{T} Peak area of fluorescein in the test solution
m_{R} Mass of fluorescein reference substance in mg in the reference stock solutions
C_{R} Declared content of the reference substance in percent
V_{T} Volume of test solution in ml
V_{D} Volume of reference stock solutions which is pipetted for further dilution in ml
PA_{R} Peak area of fluorescein in the reference solutions
m_{T} Mass of test substance in mg
V_{RS} Volume of reference stock solutions in ml
V_{R} Final volume of reference solutions in ml
Calculation C=fluorescein released in % of theoretical value

$$C = \frac{M \times 100}{{C}_{T}}$$

Where
M fluorescein released in mg/100 mg ISGS
100 Conversion factor to %
C_{T} Declared content of fluorescein in the ISGS in % mg/100 mg

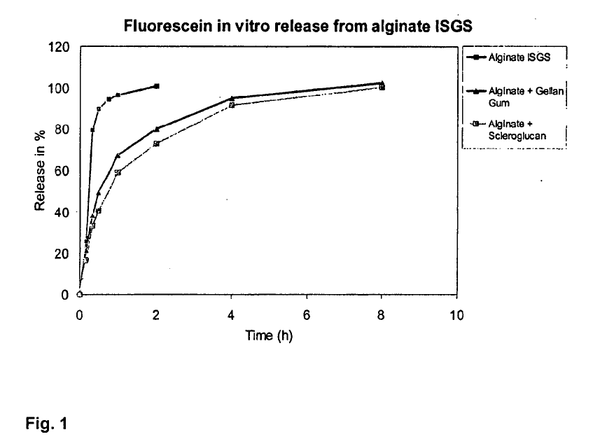

Results are depicted in FIG. 1, which shows the release profile of 0.5% w/v of fluorescein ISGVs. Release of fluorescein occurred over an 8 hour period with both alginate/gellan gum ISGVs and alginate/scleroglucan ISGVs, while release of fluorescein from a classical alginate in situ gelling system occurred in just under 2 hours. The addition of gellan gum or scleroglucan as an excipient with an alginate with a guluronic acid content of 35% to 45% increased the in vitro release time of fluorescein compared to classical alginate alone by over 4-fold.

### EXAMPLE 2

A further experiment was performed to test the effectiveness of the formulations described in Example 1 in vivo by a Schirmer test with rabbit subjects. Schirmer's test is used to determine tear production. The test is performed by placing filter paper inside the lower lid of the eye. After a few minutes, the paper is removed and tested for its moisture content. Fluorescein eye drops are also used to test if tears can flow through the lachrymal duct into the nose.

Briefly, female New-Zealand albino rabbits, weighing 3 kg to 5 kg each were supplied by Novartis animal farm and housed under standard conditions. A full ophthalmic and physical examination of the animals was performed before the beginning of the study so that only animals without pathological findings were included in the study.

A test substance of 50 μl of ISGV formulation was instilled on the outer superior part of the bulbar conjunctiva of the rabbit undergoing treatment using a gauged automatic pipette. The contralateral eye was used as non-treated control. The eyelid was gently closed for one second. Three rabbits were used per formulation, each formulation being subjected to a Schirmer test of tear sampling at 2, 4, 6, 8, and 12 hours. Tears were sampled and analyzed by HPLC. In addition, before instillation, for the first 15 minutes, 1, 2, and 7 days after instillation, ocular examinations were carried out on the rabbits using a pen lamp. Anterior segment aspect was evaluated and scored according to Draize method. Any discomfort was also scored.

Residual on Schirmer strips were tested by RP HPLC with ultra-violet detection at 225 nm. Samples of 100 μl from reference solution and test solution were aliquoted with a Rainin pipette and tested by HPLC. Equipment included an Agilent 1100 Series apparatus with a Nulceosil 100-5 C18 column 250 mm in length, with an internal diameter 4.6 mm and a flow rate of 1.0 ml/min through the column.

A system suitability test was performed prior to evaluation of Schirmer strips. Blind solution (reference) containing THF was compared to blind solution containing Schirmer strip (test solution).

Chromatograms of a reference solution were compared to test solution and peak areas were calculated to provide content of fluorescein as amount in μg per Schirmer strip. Evaluation to determine the peak area of fluorescein in the chromatograms of the test solutions and reference solutions was performed as follows:
Calculation
Assay M=Content of fluorescein in μg/Strip

$$M = \frac{{PA}_{T} \times {m}_{R} \times {C}_{R} \times {V}_{T} \times {V}_{D} \times {V}_{D}}{{PA}_{R} \times {V}_{RSS 1} \times {V}_{RSS 2} \times {V}_{RS}}$$

Where
PA_{T} Peak area of fluorescein in the test solution
m_{R} Mass of fluorescein reference substance in the reference stock solutions in mg
C_{R} Declared content of the reference substance in percent
V_{T} Volume of the test solution in ml
V_{D1} Volume of reference stock solutions 1 resp. 2 which is pipetted for further dilution in ml
V_{D2} Volume of reference stock solutions 1.1 resp. 2.1 which is pipetted for further dilution in ml
1000 Conversion factor mg to μg
PA_{R} Peak area of fluorescein in the reference solutions
V_{RSS1} Volume of the reference stock solutions in ml
V_{RSS2} Finale volume of reference stock solutions 1 resp. 2 in ml
V_{RS} Finale volume of reference solutions 1.1 resp. 2.1 in ml
C_{F} Conversion factor to mg
Assay Content of fluorescein in μg/100 mg Tears

$$C = \frac{M \times 100}{{M}_{T}}$$

Where
M Content of fluorescein in μg/Strip
100 Conversation factor to %
M_{T} Mass of tears in mg/Strip

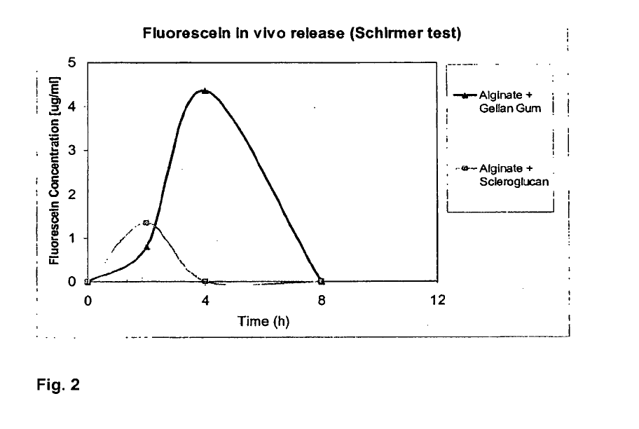

FIG. 2 shows results of the Schirmer test of 0.5% fluorescein (active agent) ISGV formulations. Fluorescein was present on the cornea of the rabbit tested for up to 30 minutes using a classical alginate gelling system. Fluorescein was detectable on the Schirmer strips of the alginate/gellan gum ISGVs up to 8 hours after instillation on the rabbit eye. Fluorescein was detectable on the Schirmer strips of the alginate/scleroglucan ISGVs up to 4 hours after the instillation on the rabbit eye. The in vivo results confirmed the in vitro studies: the optimized formulations containing alginate/scleroglucan and alginate/gellan gum showed a longer, sustained in vivo release of active agent when compared to alginate alone.

### EXAMPLE 3

Optimized test formulation compositions used to determine release kinetics are detailed in Table 1 and are prepared according to conventional laboratory methods and the following protocols.

TABLE 1
Formulation composition of ISGVs

| | Function of | Alginate | Alginate + | Alginate + |
|---|---|---|---|---|
| Name | excipient | ISGV | Gellan Gum | Scleroglucan |
| Sodium Fluorescein | Model compound | 0.5 | 0.5 | 0.5 |
| Sodium Alginate | In situ gelling | 1.5 | 1.5 | 1.5 |
| 35%-45% guluronic acid | polymer | | | |
| Gelrite (Gellan gum) | In situ gelling | | 0.03 | |
| | polymer | | | |
| Scleroglucan (tinocare | Bioadhesive | | | 0.25 |
| GL 1%) | polymer | | | |
| Hydrochloric acid 0.1N | | Ad to pH 7.4 | Ad to pH 7.4 | |
| Sodium chloride | Osmotic agent | 0.72 | 0.72 | 0.72 |
| Wfl | | Ad to 100 | Ad to 100 | Ad to 100 |

### Methods of Manufacture:

### 1.5% Sodium Alginate, 0.5% Sodium Fluorescein

In a 160 ml glass bottle about 0.50 g of sodium fluorescein was weighed and added to 95 g of nanopure water and sodium chloride. The mixture was stirred for approximately 30 minutes. Sodium alginate was added while using a RW 16 basic stirrer at 1000 rpm. Stirring occurred for 2.5 hours until complete dissolution of sodium alginate. The pH was adjusted to 7.4 with Hydrochloric Acid 0.1 N. Nanopure water was added to 100.00 g.

### 1.5% Sodium Alginate, 0.03% Gellan Gum, 0.5 % Sodium Fluorescein

In a 160 ml glass bottle 96 g of nanopure water was added prior to gellan gum, then heated for 4 h at 80° C. under stirring at 1000 rpm. The mixture was cooled down to room temperature (RT) under stirring. Sodium fluorescein (0.50 g) was added, then stirred for about 15 min. Next, sodium chloride was added while stirring for 10 min. Sodium alginate was then added while using a RW 16 basic stirrer at 1000 rpm. Stirring for 2.5 hours occurred until complete dissolution of sodium alginate. The pH was adjusted to 7.4 with hydrochloric acid 0.1 N. Nanopure water was added to 100.00 g.

### 1.5% Sodium Alginate, 0.25% Scleroglucan, 0.5% Sodium Fluorescein

In a 160 ml glass bottle, 96 g of nanopure water was added, then sodium chloride, and stirred for 10 min. Sodium fluorescein (0.50 g) was added and stirred for about 15 min. Sodium alginate was then added while using a RW 16 basic stirrer at 1000 rpm. Stirring occurred for 2.5 hours until complete dissolution of sodium alginate. Tinocare GL 1%, (25 g) was added under stirring at 1000 rpm for 10 min. Nanopure water was added to 100.00 g.

### EXAMPLE 4

A release profile similar to the release profile described in Example 1 was performed, this time using a hydrophobic model compound, ASM 981 (3% w/v) as the active agent incorporated/suspended with alginate ISGVs. Test formulations used for in vitro release studies are detailed in Table 2 below.

ISGVs containing 3% (w/v) ASM981 were incubated in aqueous release medium and underwent periodic analysis for dissolution by RP HPLC with ultra-violet detection at 210 nm. Release medium was comprised of simulated tear fluid containing glucose, NaHCO₃, adenosine and glutathione and 1% SDS. Samples of 100 μl from reference solution and test solution were aliquoted with a Rainin pipette and tested by HPLC. Equipment included an Agilent 1100 Series apparatus with a Zorbax Eclipse XDB-C18 column 150 mm in length, with an internal diameter 3.0 mm and a flow rate of 1.3 ml/min through the column. HPLC vials were Infochroma with inserts and the dissolution vessel was a 30 ml glass bottle with screw cap.

A system suitability test was performed prior to dissolution. Each batch contained between 3 and 6 samples. Samples were shaken at 60 rpm in circular motion with about 1 cm radius in a test medium of simulated tear fluid+1% SDS. Incubation involved filling the dissolution vessel with 30 ml test medium and tempering to 37° C. ASM981 test ISGV was weighed to about 300 mg accurately to 0.1 mg with an analytical balance in a weighing pan and then transferred in the dissolution vessel. The alginate containing sample was solidified within the weighing pan upon contact with the test medium. The vessel was closed and placed on a lab shaker in an oven at 60° C. Samples of 100 μl test medium were then transferred into HPLC vials and tested.

Chromatograms of a reference solution were compared to the test solution of ASM981 and peak areas were calculated to provide a release profile. Evaluation to determine the peak area of ASM981 in the chromatograms of the test solutions and reference solutions was performed as follows:
Calculation M=ASM981 released in mg/100 mg ISGS

$$M = \frac{{PA}_{T} \times {m}_{R} \times {C}_{R} \times {V}_{T} \times {V}_{D}}{{PA}_{R} \times {m}_{T} \times {V}_{RS} \times {V}_{R}}$$

where
PA_{T} Peak area of ASM981 in the test solution
m_{R} Mass of ASM981 reference substance in mg in the reference stock solutions
C_{R} Declared content of the reference substance in percent
V_{T} Volume of test solution in ml
V_{D} Volume of reference stock solutions which is pipetted for further dilution in ml
PA_{R} Peak area of ASM981 in the reference solutions
MT Mass of test substance in mg
V_{RS} Volume of reference stock solutions in ml
V_{R} Final volume of reference solutions in ml
Calculation C=ASM981 released in % of theoretical value

$$C = \frac{M \times 100}{{C}_{T}}$$

where
M ASM981 released in mg/100 mg ISGS
100 Conversion factor to %
C_{T} Declared content of ASM981 in the ISGS in mg/100 mg

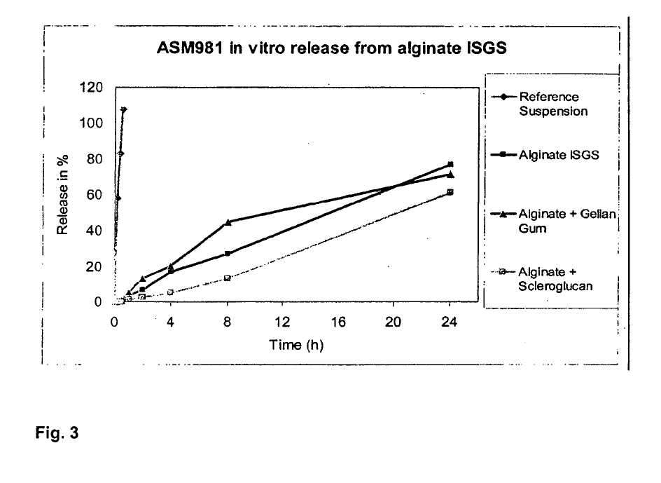

Results can be seen at FIG. 3, which shows that ASM 981−ISGV formulations have an in vitro release rate of over 24 hours.

### EXAMPLE 5

Optimized formulation compositions used for the in vitro studies of the release of a hydrophobic agent are detailed in Table 2, and are prepared according to conventional laboratory methods and the following protocols.

TABLE 2
ASM 981 ISGVs Formulation Composition:

| | Function of | Alginate | Alginate + | Alginate + |
|---|---|---|---|---|
| Name | excipient | ISGV | Gellan Gum | Scleroglucan |
| ASM981 | Model | 0.3 | 0.3 | 0.3 |
| | compound | | | |
| Sodium | In situ gelling | 1.5 | 1.5 | 1.5 |
| Alginate | polymer | | | |
| Gelrite | In situ gelling | | 0.03 | |
| (Gellan gum) | polymer | | | |
| Scleroglucan | Bioadhesive | | | 0.25 |
| (tinocare | polymer | | | |
| GL 1%) | | | | |
| Hydrochloric | | Ad to pH 7.4 | Ad to pH 7.4 | Ad to pH 7.4 |
| acid 0.1N | | | | |
| Sodium | Osmotic | 0.72 | 0.72 | 0.72 |
| chloride | agent | | | |
| Nanopure | | Ad to 100 | Ad to 100 | Ad to 100 |
| water | | | | |

### Methods of Manufacture:

### 1.5% Sodium Alginate and 0.3% ASM981

In a 200 ml glass bottle about 70 g of nanopure water is weighed, in which sodium chloride was solubilized. ASM981 was added and stirred for 15 minutes at 800 rpm, using an Ultra turrax to suspend ASM981. Sodium alginate was added and stirred for 2.5 hours using a RW 16 basic stirrer until complete dissolution of sodium alginate. The pH was adjusted to 7.0 and nanopure water was added to 100.00 g

### 1.5% Sodium Alginate, 0.03% Gellan Gum and 0.3% ASM981

In a 1000 ml glass bottle about 380 g of nanopure water was weighed, in which 3.2 g of sodium chloride was solubilized. Sodium alginate (8.002 g) was added while using a RW 16 basic stirrer at 1000 rpm. Stirring occurred for 3 hours until complete dissolution of sodium alginate was seen. Nanopure water was added to 400.00 g. In a separate 100 ml glass bottle about 20 g of nanopure water was weighed, in which sodium chloride was solubilized. Gellan gum was added and stirred for 3 hours at 800 rpm at 70° C., then cooled to room temperature (RT). ASM981 was added and stirred for 1 hour at 800 rpm. To the second glass bottle containing the gellan gum mixture, 75 g of solution A was added, using an Ultra turrax to suspend ASM981. Stirring occurred for 2.5 hours using an R W 16 basic stirrer at 1000 rpm. The pH was adjusted to 7.4. and nanopure water was added to 100 g

### 1.5% Sodium Alginate, 0.25% Scleroglucan and 0.3% ASM981

In a 200 ml glass bottle about 70 g of nanopure water was weighed, in which sodium chloride was solubilized. ASM981 was added and stirred for 15 minutes at 800 rpm using an Ultra turrax to suspend ASM981. Sodium alginate was added and stirred for 2.5 hours using a RW 16 basic stirrer until complete dissolution of sodium alginate. Scleroglucan was added and stirred for 1 hour. Nanopure water was added to 100.00 g.

The described embodiments are to be considered in all respects only as exemplary and not restrictive. The scope of the invention is, therefore, indicated by the subjoined claims rather by the foregoing description. All changes which come within the meaning and range of equivalency of the claims are to be embraced within their scope.

We claim:

1. An ophthalmic in situ-gelling vehicle for sustained drug delivery, the in situ gelling-vehicle comprising: a sodium alginate, wherein the guluronic acid content is in a range of about 35 percent to 45 percent, in combination with an excipient, and an active agent incorporated therein.

2. The in situ-gelling vehicle of claim 1, wherein sodium alginate is present in a weight percent range of about 0.5 percent to about 3 percent.

3. The in situ-gelling vehicle of claim 1, wherein the excipient comprises either gellan gum or scleroglucan, or a combination thereof.

4. An ophthalmic formulation, comprising: a sodium alginate, wherein the guluronic acid content is in a range of about 35 percent to 45 percent, and an excipient in a gelling combination for use in the sustained delivery of an active agent to the front of the eye.

5. The ophthalmic formulation of claim 4, wherein sodium alginate is present in a weight percent range of about 0.5 percent to about 3 percent.

6. The ophthalmic formulation of claim 4, wherein the excipient comprises either gellan gum or scleroglucan, or a combination thereof.

7. The ophthalmic formulation of claim 4, wherein the active agent is dissolved or suspended within the formulation.

8. A method for treating a disorder of the eye in a patient in need thereof, comprising:
administering a liquid ophthalmic formulation comprising an alginate, wherein the alginate has a guluronic acid content of about 35 percent to about 45 percent and an excipient together with a therapeutically effective amount of an active agent;
initiating gelling of the ophthalmic formulation upon instillation in the cul de sac of the eye of the patient; and
controlling release of the active agent from the gelled formulation within the eye to treat the disorder.

9. The method of claim 8, wherein the alginate has a weight percent range of about 0.5 percent to 3 percent.

10. The method of claim 8, wherein the excipient comprises either gellan gum or scleroglucan, or a combination thereof.

11. An in situ-gelling system for sustained drug delivery of an active agent to the front of the eye, the in situ-gelling system comprising: a sodium alginate with a guluronic acid content in a range of about 35 percent to about 45 percent, in combination with an excipient and an active agent.

12. The in situ-gelling system of claim 11, wherein sodium alginate is present in a weight percent range of about 0.5 percent to about 3 percent.

13. The in situ-gelling system of claim 11, wherein the excipient comprises either gellan gum or scleroglucan, or a combination thereof.
